(19) United States
(12) Patent Application Publication
(10) Pub. No.: US 2016/0250372 A1
(43) Pub. Date: Sep. 1, 2016

(54) Device for photocatalytic removal of volatile organic and inorganic contamination as well as microorganisms especially from automobile air conditioning systems

(71) Applicants: POLITECHNIKA GDANSKA, Gdansk (PL); PHU DYTRYCH SP. Z O.O., Lódz (PL)

(72) Inventors: Witold Dytrych, Lodz (PL); Adriana Zaleska, Gdansk (PL); Anna Zielinska-Jurek, Gdansk (PL); Anna Cybula, Osiek (PL); Ewelina Grabowska, Gdansk (PL); Joanna Reszczynska, Czarna Woda (PL); Michal NIschk, Olsztyn (PL); Marek Klein, Gdansk (PL); Anna Golabiewska, Gdansk (PL)

(21) Appl. No.: 14/894,704

(22) PCT Filed: Feb. 17, 2014
(86) PCT No.: PCT/PL2014/050008

§ 371 Date: Nov. 30, 2015

(30) Foreign Application Priority Data
May 31, 2013 (PL) P.404173

(51) Int. Cl. A61L 9/20

(52) U.S. Cl. CPC A61L 9/205

(57) ABSTRACT

The device for photocatalytic removal of volatile organic and inorganic contaminations as well as microorganisms especially from air conditioning systems of mechanical vehicles consisting of a plate element applied with a photocatalytic layer as well as a load-carrying element holding a light source in the form of LED diodes emitting UV light, preferably UV-A and/or UV-C, given that between the load-carrying element and the plate element created there is an arterial space, while the light source is mainly directed towards the layer in which the plate element (1) is isolated from the load-carrying element (2) with at least one spacer element (3), preferably applied with a photocatalytic layer. Preferably the external load-carrying element (2) or the external plate element (1) is equipped with a fastening element and the light source (7) consists of LEDs emitting UVis light with wavelength from 410 to 460 nm. The photocatalyst consists of nanotubes made of titanium dioxide modified with metals, preferably precious metals, obtained electro-chemically or nanocomposites made of titanium dioxide modified with metals, preferably precious metals, received using the microemulsive method.

The object of the invention is a device for photocatalytic removal of volatile organic and inorganic contamination as well as microorganisms, particularly from automobile air conditioning systems. The invention could be also applied in air conditioning systems of other mechanical vehicles and in small ventilation systems.

Known air-cleaning devices can be divided according to the volume of the stream of purified into air ventilation and air-conditioning devices assembled in process halls and rooms with large traffic intensity as well as small air-conditioning and ventilation devices, among which we can distinguish devices intended for degradation of contamination in automobile air conditioning systems.

Application specification U.S. Pat. No. 5,835,840 depicts a system for photocatalytic purification of air inside rooms. Inside the reactor is covered with a layer of titanium dioxide. The recommended air humidity is 50%. A thin layer of titanium dioxide was applied on the internal surface of the ventilation duct. Inside the ventilation duct there is a set of lamps emitting ultraviolet light with the wave length of (=300÷400 nm).

Patent documentation US007255831B2 depicts a system for photocatalytic purification of air in closed rooms. The applied photocatalyst is made of titanium dioxide modified with tungsten (VI) monoxide. The photocatalyst modified with tungsten was received by mixing a water solution (NH₄)₁₀ W₁₂ O₄₁ with a TiO₂</0 suspension>. Next, the obtained photocatalyst was dispergated in water in the amount of 25% of weight and applied on the surface of a substrate with a honeycomb structure by electrophoretic spraying or immersion (dip-coating) An important stage in the process of formation of thin layers of the photocatalyst is the process of homogenisation of WO3/TiO₂ nanoparticles in water, which should last between 10 and 30 minutes. As a result, homogeneous layers are obtained by carrying out a single process of impregnation of the surface of the substrate. The photocatalytic activity was examined in a degradation reaction of acetylaldehyde and 1-propanol in the gaseous phase. The highest photocatalytic activity was demonstrated by the WO₃ /TiO₂ photocatalyst for air humidity amounting to 40-50%, while non-doped TiO₂ for air humidity amounting to 20%. However, in the case of degradation of butene in the presence of the 3% mol. WO₃/TiO₂ photocatalyst it is beneficial to reduce the air humidity down to 20%.

Patent specification US006797127B1 describes an air-cleaning system consisting of UV light sources. The first stage was installation of a lamp emitting radiation with the wave length ranging from 110 to 200 nm to generate ozone. The second and the third stage of the air purification process involves emission of radiation with the wave length of 200-300 nm and 300-380 nm, while the process of degradation of contamination occurs in the presence of a photocatalyst of titanium dioxide with an orthorhombic structure modified with silver, gold, platinum, tungsten, vanadium or copper. The photocatalyst was set on the surface of a metal tile, a ceramic base, polyester fibres, paper, plastic or paper filter.

Application documentation US005762665A describes a an air-cleaning device for automobile ventilation systems consisting of passive (horizontal barriers) and active (carbon filters) filters. A system of horizontal barriers forces air flow. Anti-dust filters made of carbon fibres are placed in a rectangular casing inside the space between the passive filters. Air circulation is forced by a fan.

Application specification US2007/0032186A1 depicts an air-cleaning system installed in cabins consisting of a system of five filters. The first one removes particles bigger than 10 μm, the second one carbon filter stops dirt present in the gaseous phase, additionally the filtration material contains activated aluminium oxide impregnated with permanganate, the third filter removes solid particles sized 2.5 μm and smaller. The fourth photocatalytic filter is used for removal of pathogenic bacteria and is made in the form of titanium plate. The air flow efficiency in the designed system is at least 400 m³/min.

Application specification US20110105008A1 depicts an air-cleaning system for vehicles involving pre-cleaning heated air from the engine and the turbocharger. The catalytic system air purification is placed between the mixing valve and the closed passenger cabin. The catalytic contamination removal unit (CATOX) works in the range of temperatures between 500° F. and 830° F.

Application specification US2012/0128539A1 describes a an air-cleaning device constituting an integral part of the automobile ventilation system. The internal part of the ventilation duct is covered with a layer of the photocatalyst. An UVA-emitting lamp or a system of UVA-emitting diodes was used as the light source.

In order to avoid inconveniences related with the surfaces of the lamps becoming dirty known devices are assembled in systems behind the filters absorbing solid dirt particles. Nevertheless, the dirt on the lamps causes a non-uniform delivery of the radiation to the surface of, the photocatalyst, which causes the efficiency of dirt oxidation to diminish. In consequence, replacement of the entire air conditioning system may become necessary.

A device for photocatalytic removal of volatile organic and inorganic contamination as well as microorganisms, particularly from air conditioning systems of motor vehicles consisting of a plate element covered with a photocatalytic layer as well as a load-carrying element supporting a light source consisting in the form of LEDs emitting UV light, preferably UV-A and/or UV-C, given that there is an arterial space between the load-carrying element and the plate element, while the light source is directed towards the photocatalytic layer is characterised according to the invention by the fact that the plate element is isolated from the load-carrying element by at least one spacer, preferably covered with a photocatalytic layer. The photocatalytic layer is made of the photocatalyst applied by following a known method uniformly and/or at certain points.

In a variant of the invention the spacer is made in the form of a net.

In other variant of the devices according to the invention the spacer is shaped in the form of a corrugated section of the side wall of a cylinder.

In another variant of the invention the distance of the photocatalytic layer from the light source ranges from 1 to 30 cm, preferably from 2 to 7 cm.

In one other variety of the invention the load-carrying or the external plate element is equipped with a fastening element and possibly a sealing element.

Preferably, the external load-carrying element or the external plate element is equipped with a distance cover plate.

In the next variant of the device according to the invention the light source additionally consists of LEDs emitting UVis light with the wave length from 410 to 460 nm, preferably 410-430 nm.

Preferably, the ratio of the number of diodes emitting UV-A:UV-C: UVis light ranges from 1:1:1 to 1:1:8, preferably 1:1:4, given that preferably at most 20% LEDs are set in the way that the light is thereby emitted by them at an angle of 15 to 75° in relation to the load-carrying element.

In the next variant of the device according to the invention UV-C light intensity amounts to from 0.5 to 25 mW/cm², preferably from 2 to 8 mW/cm², UV-A light intensity amounts to from 0.5 to 25 mW/cm², preferably from 2 to 8 mW/cm ², while UVis light intensity amounts to from 0.5 to 25 mW/cm², preferably from 2 to 8 mW/cm².

In another variant of the invention the spacing elements are preferably placed straight-through the load-carrying element and ended on both sides with plate elements, given that the light source is placed on both sides of the load-carrying element.

In another variety of the invention the spacing elements are preferably placed straight-through the plate and ended on both sides with load-carrying elements, given that the photocatalytic layer is located on both sides of the plate element.

In another variant of the invention the spacing elements are preferably placed straight-through in the internal load-carrying element and ended on one side with an external plate element and on the other with an external load-carrying element.

Preferably, the photocatalyst consists of titanium dioxide nanotubes modified with metals, preferably precious metals received as a result of an electro-chemical reaction.

Preferably, the photocatalyst consists of titanium dioxide nanocomposites modified with metals, preferably precious metals received by using the micro emulsive method.

The stream of purified air is introduced in parallel or perpendicularly to the photocatalytic layer contained in the device according to the invention. The device is intended for installation in an existing automobile air conditioning duct as a standalone element or an element of a multi-element system forming a battery of devices. Should the lamps become dirty, it will be sufficient to replace the device itself, without the need to replace the entire or part of the air conditioning system in the car. Using UVis radiation reduces power consumption the amount of which in motor vehicles is limited, conditioned by the capacity of the battery.

The subject of the invention has been explained in more detail in the implementation examples and in the figure where:

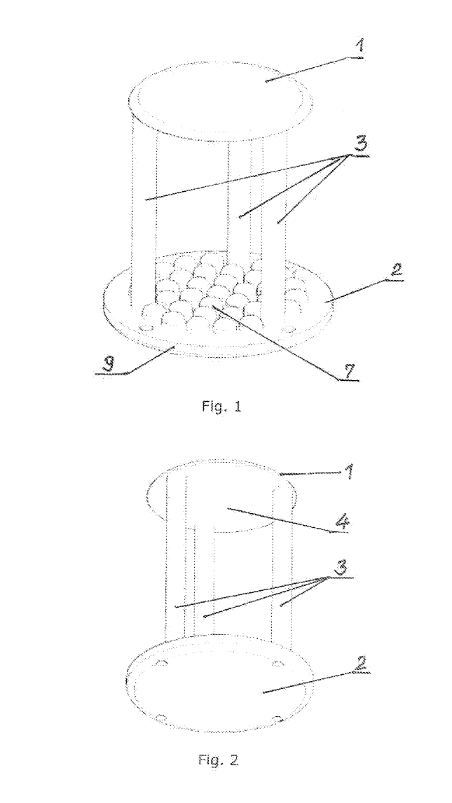

FIG. 1 presents a variety of the device viewed from the side of the plate element,

FIG. 2 presents the variety of the device from FIG. 1 viewed from the side of the load-carrying element,

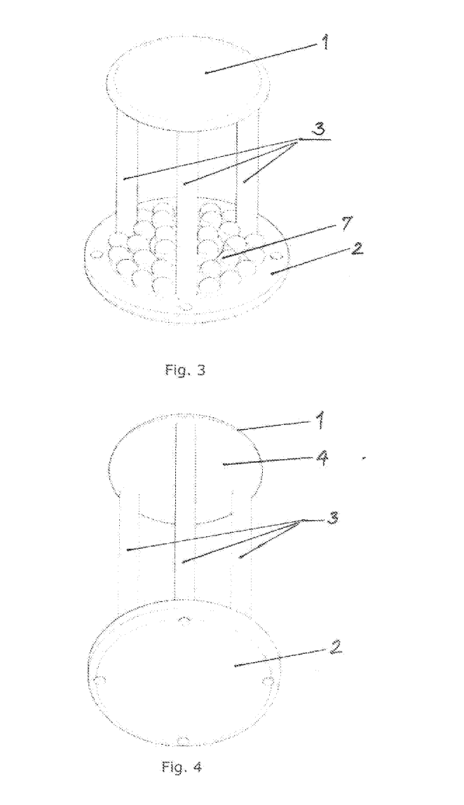

FIG. 3 presents a variety of the device in which the radiation from some LEDs falls on the photocatalytic layer at an angle viewed from the side of the plate element,

FIG. 4 presents the variety of the device from FIG. 3 viewed from the side of the load-carrying element,

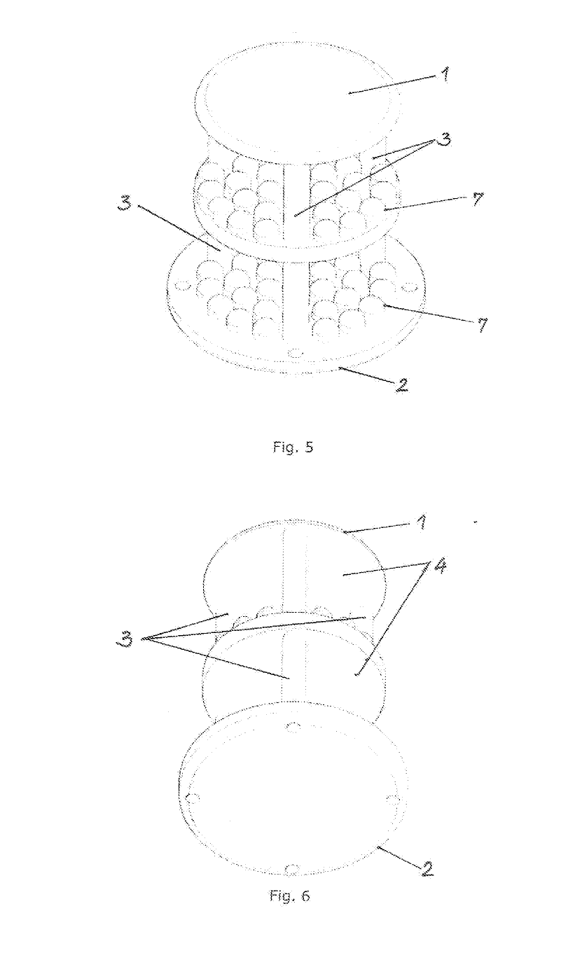

FIG. 5 presents a variety of the device with two photocatalytic layers viewed from the side of the plate element,

FIG. 6 presents the variety of the device from FIG. 5 viewed from the side of the load-carrying element,

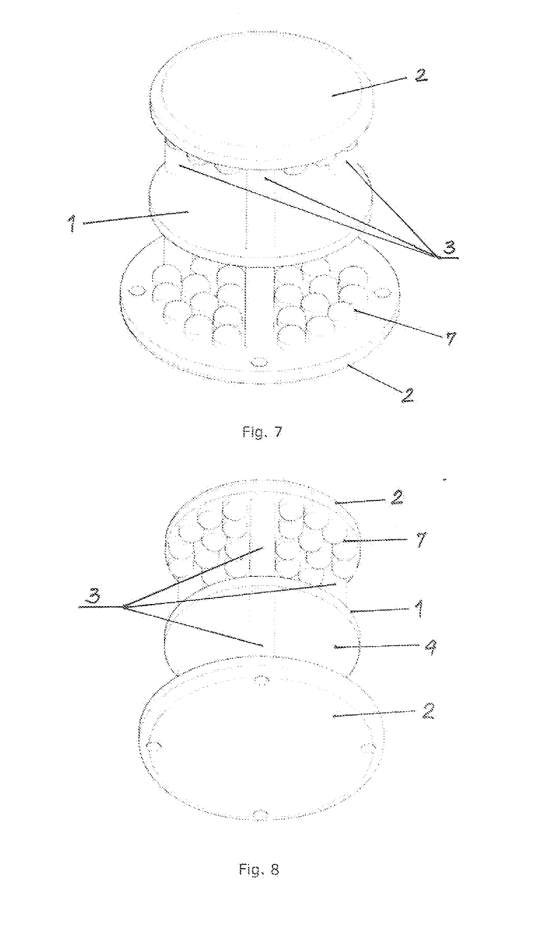

FIG. 7 presents a variety of the device with two photocatalytic layers viewed from the side of the load-carrying element,

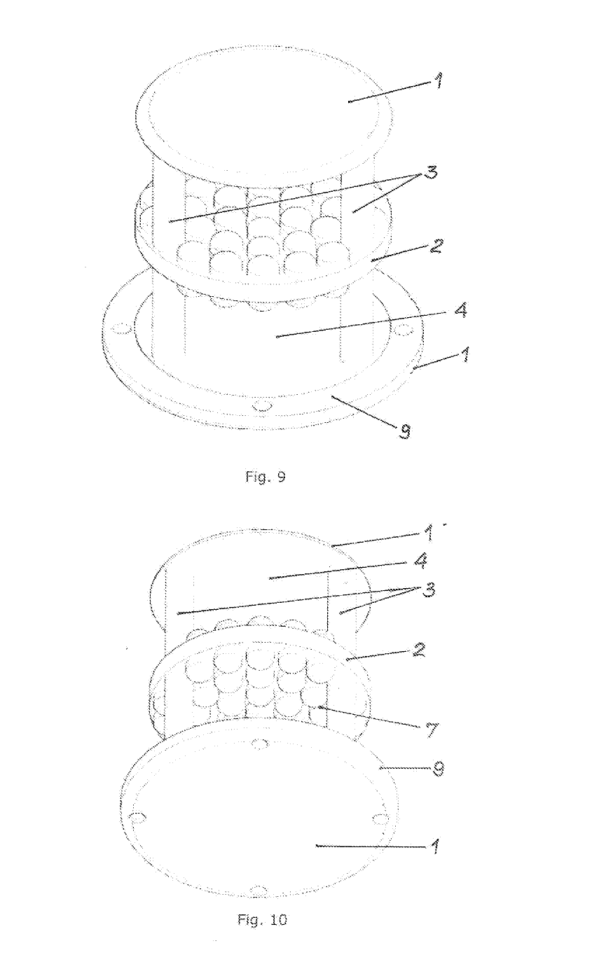

FIG. 8 presents the variety of the device from FIG. 7 viewed from the side of the second load-carrying element,

FIG. 9 presents a variety of the device with the load-carrying element with the light sources placed on both sides viewed from the side of the plate element,

FIG. 10 presents the variety of the device from FIG. 7 viewed from the side of the second load-carrying element,

FIG. 11 presents a variety of the invention with a spacer in the form of a section of the side wall of a cylinder viewed from the side of the plate element,

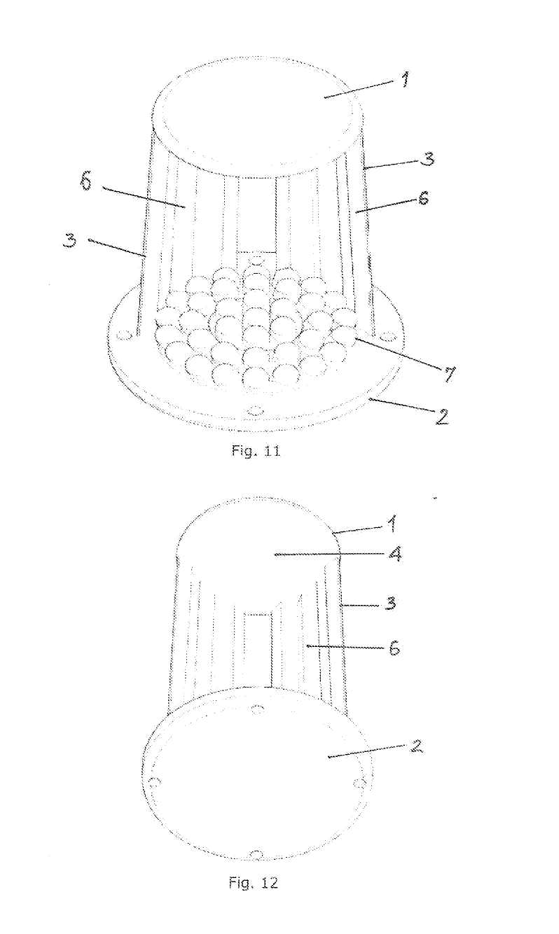

FIG. 12 presents the variety of the device from FIG. 11 viewed from the side of the load-carrying element,

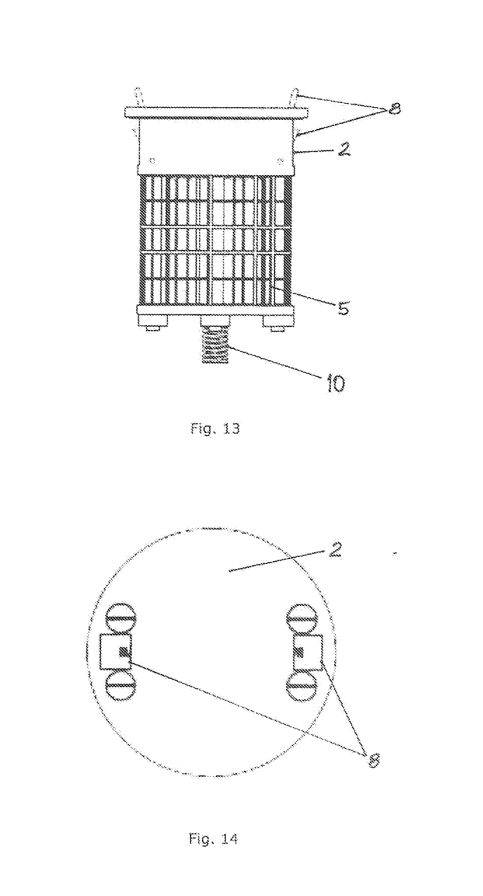

FIG. 13 and FIG. 15 present a variety of the device with a spacer made in the form of a net viewed from the back,

FIG. 14 presents the device from FIG. 13 viewed from the top,

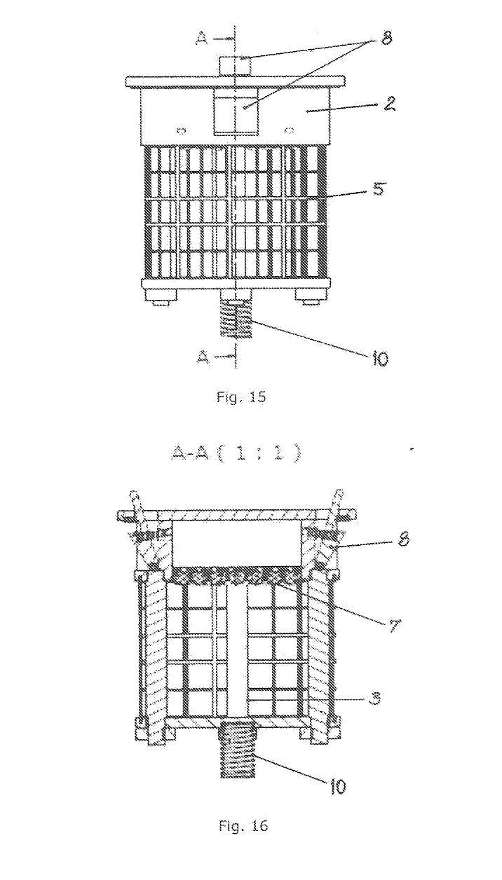

FIG. 16 presents the device from FIG. 15 in a vertical section,

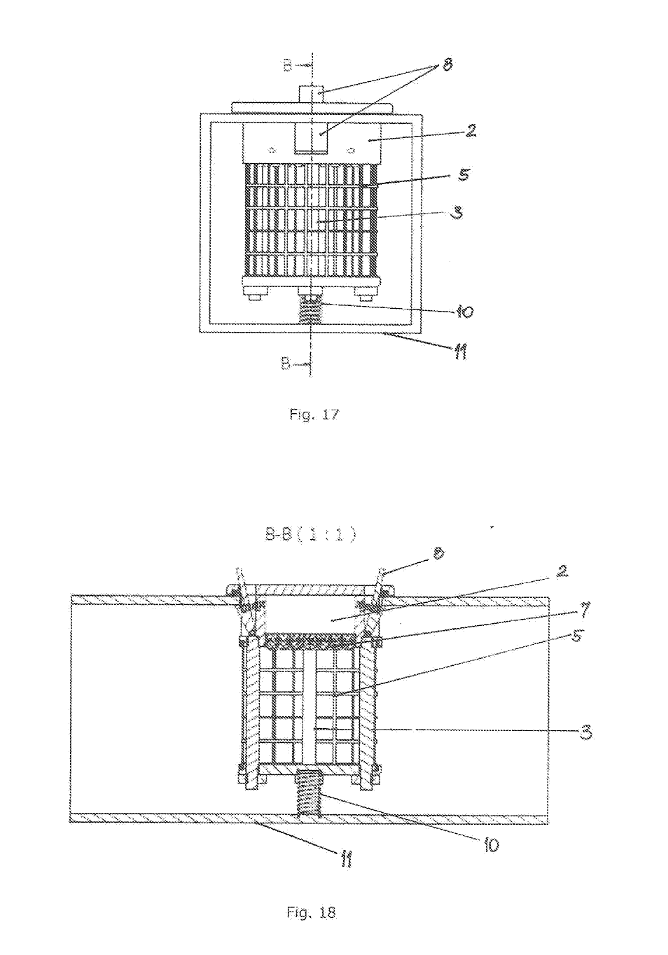

FIG. 17 presents the device from FIG. 13 placed in the air conditioning duct viewed in the transverse section of the duct

FIG. 18 presents the device from FIG. 13 placed in the air conditioning duct in the longitudinal section of the duct.

### EXAMPLE I

### Obtaining the Photocatalytic Layer

350 cm³ 0.2M AOT (sodium bis-2-ethylhexyl sulfosuccinate) in cyclohexane were added with 1.44 cm ³ of a water solution of silver nitrate and potassium hexachloroplatinate (IV) in the molar ratio of 0.5:0.1. The whole mixture was mixed for 30 min in the neutral gas atmosphere and then introduced with a precursor of titanium dioxide tetraisopropyl titanate (TIP). The microemulsion containing metal ions in the internal phase was added with 150 cm³ of microemulsion prepared as a solution of 0.2M AOT (sodium bis-2-ethylhexyl sulfosuccinate) in cyclohexane containing sodium borohydride in a dispergated phase as the reducing reagent. A 3-times excessive amount of the reducing agent in relation to the amount of moles of the metals was applied. The received Pt/Ag nanocomposites were separated, rinsed with acetone and water, dried at the temperature of 80° C. and calcinated at the temperature of 350° C. for 3 hrs. The obtained structures were bimetallic alloy-type structures set on the surface of the titanium dioxide.

As shown on FIG. 1, photocatalytic layer 4 on the basis of titanium dioxide modified with precious metals is placed on plate element 1 on the opposite side of light source 7 placed on load-carrying element 2. Photocatalytic layer 4 on the basis of titanium dioxide has photocatalytic properties with regard to the UV and the Vis electromagnetic spectrum as a result of modification of the surface of the titanium dioxide with precious metals. Plate element 1 is connected to load-carrying element 2 with spacers 3. The received photocatalyst is applied in the form of a suspension with a brush on the surface of a glass mat impregnated earlier with tetraethyl orthosilicate (TEOS) The mat was dried at the temperature of 80° C. and then placed on plate element 1. Light source 7 is made up of a system of combined LEDs, light intensity: 6 mW/cm²⅔ of which emits UV and ⅓ emits radiation from the visible light range (UVis) The device is clicked in perpendicular into air conditioning duct 11 using fastening element 8. Load-carrying element 1 is equipped with seal 9. The whole volume of purified air flows between light source 7 and photocatalytic layer 4.

When the linear flow speed of the air stream was 0.3 m/s and the humidity 30%, the degree of degradation of toluene after 20 minutes of exposure was 100%.

### EXAMPLE II

### Obtaining the Photocatalytic Layer

Photocatalytic layer 4 on the basis of TiO₂ is modified with platinum. The platinum content is 0.1% of the weight in relation to the mass of TiO₂ . The photocatalyst was obtained as a result of adding a water solution of potassium haxeachloroplatinate (IV) to isopropyl alcohol. Next, the substance was added with a precursor of titanium dioxide tetraisopropyl titanate (TIP). The received sol was dried at the temperature of 80° C. and calcinated at the temperature of 450° C. for 3 hrs. Next, the photocatalyst was set using the method of immersion on the surface of a ceramic material and placed in the device as plate element 1. LEDs UV and Vis-emitting LEDs, intensity: 8 mW/cm² were used as light source 7. As shown in FIG. 9, spacing elements 3 were set straight-through load-carrying element 2 and ended on both sides with plate elements 1. Light source 7 is placed on both sides of the load-carrying element 2.

The reactor was placed in perpendicular into air conditioning duct 11 and screwed in on thread 8. The whole volume of purified air flows in circulation between light sources 7 and photocatalytic layer 4.

When the linear flow speed of the air stream was 0.2 m/s and the humidity 50%, the degree of degradation of toluene after 30 minutes of exposure was 95%.

### EXAMPLE III

### Obtaining the Photocatalytic Layer

500 cm³ of 0.2 M AOT in cyclohexane were added with 0.5 cm³ of a water solution of potassium haxeachloroplatinate (IV) and tetrachloroauric acid (III). The whole mixture was mixed in the neutral gas atmosphere and then was added with a second microemulsion containing a reducing reagent—sodium borohydride dispergated in the water phase of a water-AOT-cyklohexane microemulsion. A threefold excessive amount of the reducing agent in relation to the amount of moles of the metals was applied. TiO₂ nanocomposites were separated and dried at the temperature of 80° C. and calcinated at the temperature of 450° C. The content of metals was 0.1% mol of Pt and 0.5% mol of Au in relation to the amount of TiO₂ moles.

Photocatalytic layer 4 made of TiO₂ modified with Au and Pt is applied in the form of a suspension with a brush on the surface of a glass mat impregnated earlier with tetraethyl orthosilicate (TEOS) and placed as shown in FIG. 11 in the air-cleaning reactor on spacer 3 shaped in the form of a section of the side wall of cylinder 6 folded like a concertina and on plate element 1. Light source 7 is made up of a system of combined LEDs, light intensity: 6 mW/cm²⅔ of which emits UV and ⅓ emits radiation from the visible light range—UVis. 20% of LEDs are placed on load-carrying element 2 in the way that they emit light at the angle of 30° in relation to plate element 1, so that spacer 3 is also illuminated. The reactor is put in perpendicular to the air flow inside the ventilation duct.

When the linear flow speed of the air stream was 0.25 m/s and the humidity 40%, the degree of degradation of toluene after 40 minutes of exposure was 100%.

### EXAMPLE IV

### Obtaining the Photocatalytic Layer

The cleaned surface of titanium steel sheet was placed in the solution of ethylene glycol (98% of vol.), water (2% vol.) and ammonium fluoride (0.09 M). The solution was placed in a vessel made of plastic. The base material was placed in the solution vertically, in such a way that only ⅔ of its amount was submerged. The electro-chemical process was conducted for 60 minutes. During the process the solution was constantly mixed. During the electro-chemical process TiO₂ nanotubes, diameter: 200 nm and length: 6 μm, were formed on the surface of the base material. The base material along with the matrix of TiO₂ nanotubes formed on the surface were taken out of the solution, rinsed with demineralised water and then placed in demineralised water and subjected to ultrasounds for 5 min. The received material was dried in 80° C. for 24 hrs. and calcinated at the temperature of 450° C. for 6 hrs. (using a temperature increase of 2° C/min). Next, the base with the nanotubes is immersed in a solution of water and isopropanol (1:1) containing potassium hexachloroplatinate (0.05M); the pH of the solution was 5. Then air was removed by passing argon through for 30 min.; the solution was mixed for 120 min in order to provide adsorption of metal ions on the surface of the nanotubes and irradiated with radiation with the wave length of 300-400 nm for 60 min.

The received material was dried at the temperature of 80° C. for 2 hrs and then applied on grid 5 made of wire , thickness: 1 mm and 3 mm diagonal mesh, constituting spacer 3. Light sources 7 in the form of a combination of LEDs emitting UV-A radiation with the wave length of 375 nm, UV-C with the wave length of 254 nm and Vis with the wave length of 415 nm, intensity: 8 mW/cm², in mutual ratio of 1:1:4, was placed on external load-bearing elements 2. 10% of the LEDs were placed at the angle of 45%, so that they would illuminate photocatalytic layer 4 on net 5. One of the external load-bearing elements is equipped with fastening element 8 and the other in a distance cover plate 10.

When the linear flow speed of the air stream was 0.25 m/s and the humidity 20%, the degree of degradation of toluene after 20 minutes of exposure was 100%.

1. A device for photocatalytic removal of volatile organic and inorganic contamination as well as microorganisms, particularly from air conditioning systems of motor vehicles consisting of a plate element covered with a photocatalytic layer as well as a load-carrying element supporting a light source consisting in the form of LEDs emitting UV light, preferably UV-A and/or UV-C, given that between the load-carrying element and the plate element there is an arterial space, while the light source is directed towards the photocatalytic layer characteristic by the fact that plate element (1) from the load-carrying element (2) is isolated by at least one spacer (3), preferably covered with a photocatalytic layer (4), given that the photocatalytic layer (4) is made of the photocatalyst applied by following a known method—uniformly and/or at certain points.

2. The device according to claim 1 is characteristic by the fact that spacer (3) has the form of a net (5).

3. The device according to claim 1 is characteristic by the fact that spacer (3) is shaped in the form of a corrugated section of the side wall of a cylinder (6).

4. The device according to claim 1 or 2, or 3 is characteristic by the fact that the distance of the photocatalytic layer (4) from the light source (7) ranges from 1 to 30 cm, preferably from 2 to 7 cm.

5. The device according to claim from 1 to 4 is characteristic by the fact that the external load-carrying element (2) or the external plate element (1) is equipped with a fastening element (8) and possibly a sealing element (9).

6. The device according to claim from 1 to 4 is characteristic by the fact that the external load-carrying element (2) or the external plate element (1) is equipped with a distance cover plate (10).

7. The device according to claim from 1 to 6 is characteristic by the fact that the light source (7) additionally consists of LEDs emitting UVis light with the wave length from 410 to 460 nm, preferably 410-430 nm.

8. The device according to claim 7 is characteristic by the fact that the ratio of the number of diodes emitting UV-A: UV-C: UVis light ranges from 1:1:1 to 1 1:8, preferably 1:1:4, given that preferably at most 20% of LEDs are set in the way that the light is thereby emitted by them at an angle of 15 to 75° in relation to the load-carrying element.

9. The device according to claim 7 or 8 is characteristic by the fact that UV-C light intensity amounts to from 0.5 to 25 mW/cm², preferably from 2 to 8 mW/cm², UV-A light intensity amounts to from 0.5 to 25 mW/cm ², preferably from 2 to 8 mW/cm², while UVis light intensity amounts to from 0.5 to 25 mW/cm², preferably from 2 to 8 mW/cm².

10. The device according to claim from 1 to 7 is characteristic by the fact that the spacing elements (3) are preferably placed straight-through the load-carrying element (2) and ended on both sides with plate elements (1), given that the light source (7) is placed on both sides of the load-carrying element (2).

11. The device according to claim from 1 to 7 is characteristic by the fact that the spacing elements (3) are preferably placed straight-through the plate element (1) and ended on both sides with load-carrying elements (2), given that the photocatalytic layer (4) is located on both sides of the plate element (1).

12. The device according to claim from 1 to 7 is characteristic by the fact that the spacing elements (3) are preferably placed straight-through in the internal load-carrying element and ended on one side with an external plate element (1) and on the other with an external load-carrying element (2).

13. The device according to claim from 1 to 12 is characteristic by the fact that the photocatalyst consists of titanium dioxide nanotubes modified with metals, preferably precious metals received as a result of an electro-chemical reaction.

14. The device according to claim from 1 to 12 is characteristic by the fact that the photocatalyst consists of titanium dioxide nanocomposites modified with metals, preferably precious metals received by using the microemulsive method.
